(19) United States
(12) Patent Application Publication
(10) Pub. No.: US 2014/0121649 A1
(43) Pub. Date: May 1, 2014

(54) WOUND DRESSING ASSEMBLY WITH ABSORBENT LAYER

(71) Applicant: Wayne A. Calco, Aliso Viejo, CA (US)

(72) Inventor: Wayne A. Calco, Aliso Viejo, CA (US)

(21) Appl. No.: 14/064,076

(22) Filed: Oct. 25, 2013

Related U.S. Application Data
(60) Provisional application No. 61/719,452, filed on Oct. 28, 2012.

(51) Int. Cl. A61F 13/02; A61M 27/00

(52) U.S. Cl. CPC A61F 13/0216; A61M 27/00
USPC 604/543

(57) ABSTRACT

A wound dressing assembly for a surgical tube or other device entrance into the body includes a cover layer, with adhesive on the outer perimeter of the underside of the cover layer to attach the wound dressing to the patient's body, an absorbent ring secured to the underside of the cover. An interior clear window allows the nurse or other medical personnel to view the wound site through the window for inspection for infection or bleeding. A port is formed in the window area to allow a tube or other device to pass through the wound dressing.

### CROSS-REFERENCE TO RELATED APPLICATION

This application claims the benefit of U.S. Provisional Application No. 61/719,452, filed Oct. 28, 2012, the entire contents of which are hereby incorporated by reference.

### BACKGROUND

Some surgical procedures involve or result in tubes being passed through the skin or wound of the patient to the interior of the patient's body. The tube may be for drainage, for example. Dressing the patient at the entrance of the tube into the body is time consuming, typically involving application of layers of gauze and adhesive tape, to construct a dressing that will seal around the tube. Such dressings cover the wound/tube entrance into the body with opaque dressing layers, e.g. gauze, and the medical personnel caring for the patient cannot see the wound area to monitor for infection, bleeding and other problems.

### BRIEF DESCRIPTION OF THE DRAWINGS

Features and advantages of the disclosure will readily be appreciated by persons skilled in the art from the following detailed description when read in conjunction with the drawing wherein:

FIG. 1 is an isometric diagrammatic view of an exemplary embodiment of a surgical dressing assembly in place on a patient's body and with a chest tube protruding through an integrated port of the dressing assembly.

FIG. 2 is an exploded isometric view of the surgical dressing of FIG. 1.

FIG. 3 is an isometric view of the dressing of FIG. 2, illustrating an overlap portion of the dressing assembly.

FIG. 4 is a cross-sectional view of the surgical dressing assembly of FIG. 3, taken along line 4-4 of FIG. 3.

FIG. 5 is a top view of an alternate embodiment of a surgical dressing with an integrated tube port.

FIG. 6 is an isometric view of another embodiment, illustrating an optional tube support structure in place over the surgical dressing assembly of FIG. 1 on the patient's body, with the tube protruding through the dressing assembly and the tube port in the tube support structure.

FIG. 7 is a top view of the tube support structure of FIG. 6, showing the perimeter of a cotton gauze element which is part of the surgical dressing assembly.

FIG. 8 is a diagrammatic isometric view of the tube support structure of FIG. 7.

FIG. 9 is a diagrammatic cross-sectional view taken along line 9-9 of FIG. 8.

FIG. 10 is a top view illustrating a further embodiment of a surgical dressing with an integrated tube port.

FIGS. 11A, 11B and 12 illustrate another embodiment of a wound dressing.

FIGS. 13A and 13B illustrate another embodiment of a wound dressing.

### DETAILED DESCRIPTION

In the following detailed description and in the several figures of the drawing, like elements are identified with like reference numerals. The figures are not to scale, and relative feature sizes may be exaggerated for illustrative purposes.

An exemplary embodiment of a surgical wound dressing for a surgical tube wound or other device entrance into the body includes a round dressing structure, with adhesive on the outer perimeter of the underside, an absorbent gauze ring secured to the underside by an annular portion of the adhesive, and an interior clear window allowing the nurse or other medical personnel to see if there is infection or bleeding. At the very center the window area may be sliced like a pie to allow a tube to pass through the wound dressing. The dressing can be fabricated in multiple sizes, and in shapes other than round. In an exemplary embodiment, the dressing is also sliced from the center all the way to the outside allowing the nurse to apply the dressing to a wound site with an existing tube exiting the human body, by opening or spreading the dressing apart at the slice or slit sufficiently to pass the dressing around the tube. A secondary molded plastic structure may optionally be included, to fit over the dressing and support the tube itself.

The dressing assembly will typically be fabricated using sterile materials, and provided for use in a sterile packaging. The nurse or medical personnel dressing the patient wound site opens the sterile packaging, removes a release layer covering the outer portion of the adhesive on the dressing to expose the adhesive ring at the outer perimeter of the dressing, and positions it on the patient's body, opening the dressing at a radial slit to position the dressing around the existing tube exiting the patient's body. The adhesive holds the dressing assembly in place on the patient's body. The radial slit is sealed by an overlap portion of the top cover layer, or by a separate tape portion. The window permits visual inspection of the wound site without removing the dressing.

Embodiments of the dressing assembly may include one or more of the following advantages or features. The dressing assembly provides ease of use, is self-contained, and saves valuable time in dressing the wound site or tube entry location. This benefits the patient as well as the medical personnel.

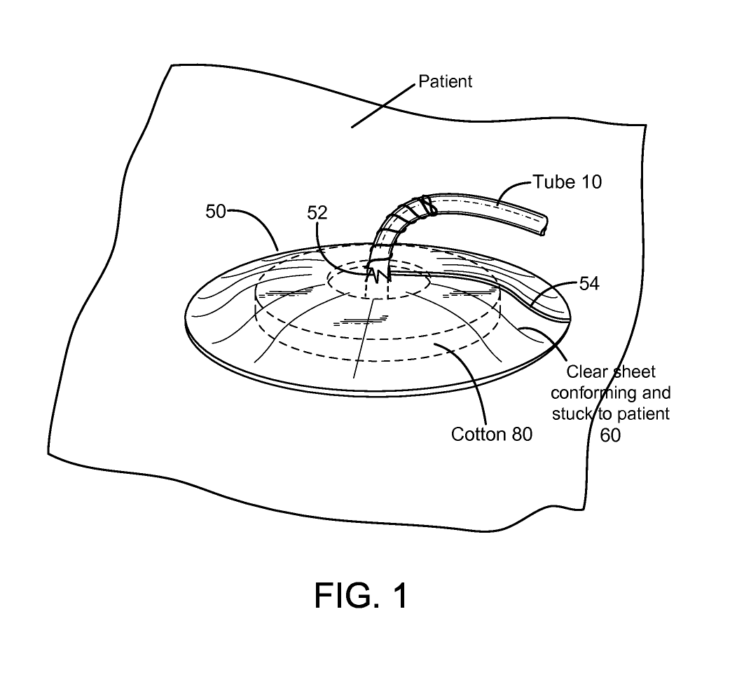

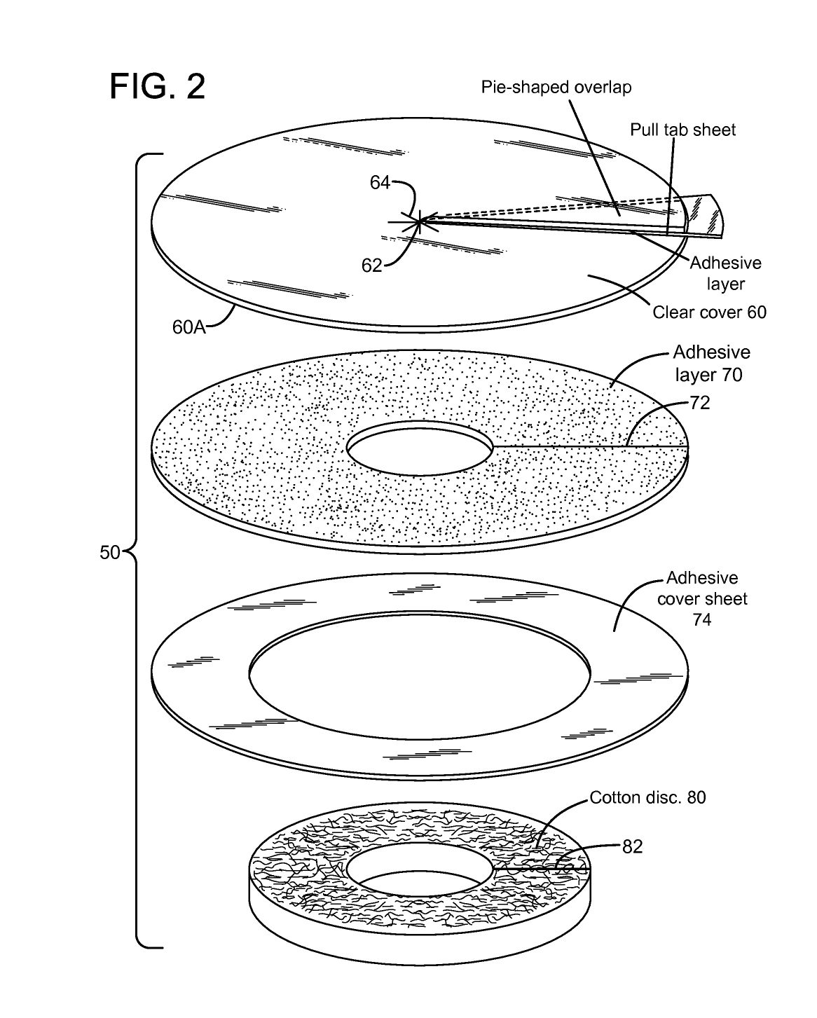

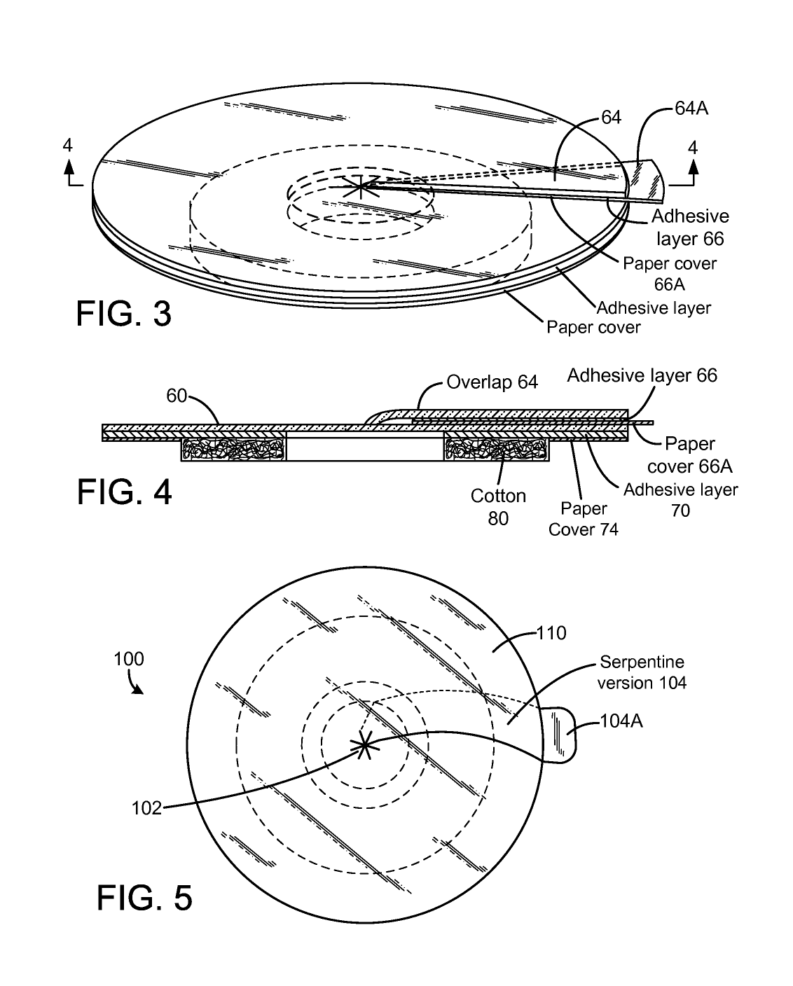

An exemplary dressing assembly 50 is illustrated in FIGS. 1-4. In this embodiment, the assembly has a generally circular configuration, but may be fabricated in other configurations, e.g. rectangular or trapezoidal. FIG. 1 diagrammatically depicts the dressing assembly 50 in position on a patient's wound site, with an existing tube 10 exiting the body and protruding from the wound site, through a dressing tube port 52. In this embodiment, the dressing assembly includes a top clear cover layer 60, which may be fabricated of a flexible plastic material or film. At or adjacent the center of the cover layer, a port 62 is defined by several spaced radial slits 64 cut through the cover layer. Alternatively, the port may be formed by a hole cut through cover layer of a diameter or size approximately the same diameter as the tube or other device exiting the body. For example, the dressing may also be suitable for application to the body for wiring connected to devices implanted within the body.

In an exemplary embodiment, the dressing assembly further includes an adhesive layer 70 adhered to the underside 60A of the cover layer 60. In this exemplary embodiment, the adhesive covers the outer peripheral portion of the underside of the cover layer, leaving open a center region of the cover layer. The outer portion of the adhesive layer is covered with a cover sheet or release layer 72, which is removed before the dressing is placed onto the patient's body. The interior portion of the adhesive functions to hold in place a ring-shaped layer or disc 80 of absorbent material, such as cotton gauze.

The dressing assembly 50 is provided, in this exemplary embodiment, with a slit 54 formed from the tube port 52 through to the edge of the assembly. In an exemplary embodiment, the slit 54 defines opposed radial edges in the cover layer, adhesive layer and absorbent material. The slit allows the dressing assembly to be opened at the slit edges enough to allow the dressing to be passed around the tube 10, as the dressing is being applied to the patient. With the dressing in position surrounding the tube, the edges can be brought together, or even overlapped, and the exposed cover layer periphery adhered to the patient's skin by the exposed portion of the adhesive. The adhesive layer 70 and the absorbent ring 80 may each have slits 72, 82, respectively, in registration with the slit edge formed in the cover sheet. In one exemplary embodiment, the cover layer 60 is formed with an overlap flap portion 64 and extended tab portion 64A. A separate pie shaped adhesive layer 66 may be applied to the underside of the overlap flap portion 66 and covered with a similarly shaped cover or release sheet 66A, to be used to seal the overlap flap portion 66 to the underlying portion of the cover layer to seal the slit. The adhesive cover sheet 74 may or may not have a slit formed through it; the lack of a slit may provide some benefit in holding together the dressing assembly prior to application to a patient.

FIG. 5 is a top view illustrating an alternate embodiment of a wound dressing assembly 100, similar to wound dressing 50, except that the slit 104 which allows the dressing to be opened and fit around the patient tube is curved or serpentine, rather than being essentially linear as with the embodiment of FIGS. 1-5.

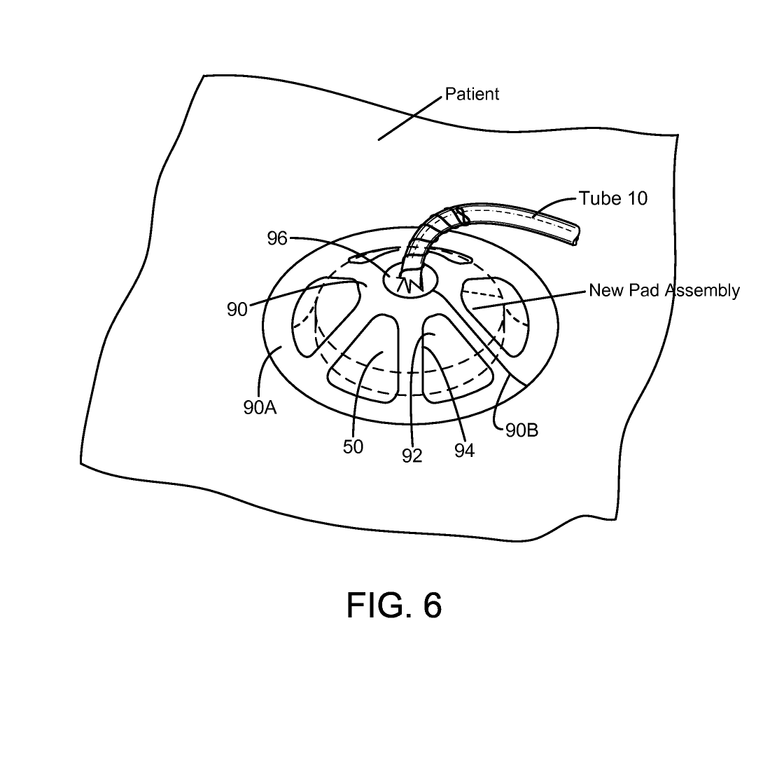

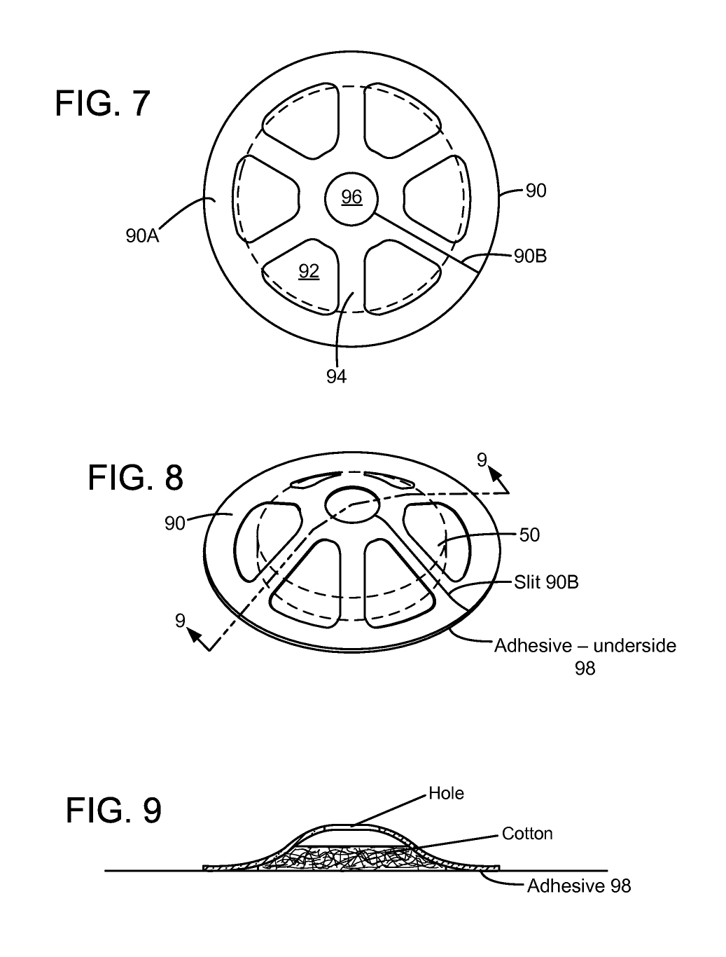

FIGS. 6-9 illustrate an optional tube support structure 90, which may be used in combination with the wound dressing assembly 50 or 100. The structure 90 has a dome-like center configuration with a tube port 94. Web ribs 94 extend from an outer generally flat periphery 90A to an interior portion in which the port is formed, with open window areas 94 defined between the ribs. This allows the wound site to remain visible through the clear cover layer of the wound dressing 50, when the structure 90 has been positioned over the dressing 50. The structure 90 includes a slit 90B extending from the port to the periphery, to allow the structure to be opened sufficiently to fit around the existing tube in the patient's body, and then adhered to the dressing periphery or patient skin by an adhesive layer 98. A cover layer may be used to cover the adhesive layer until the structure is ready for use.

The tube support structure 90 may be fabricated of a clear or opaque plastic material, having sufficient flexibility to be spread apart at the slit 90B to fit around the tube, yet rigid enough to provide some support for the tube and protection for the wound site. In an exemplary embodiment, the tube support structure 90 may be injection molded from polyethylene.

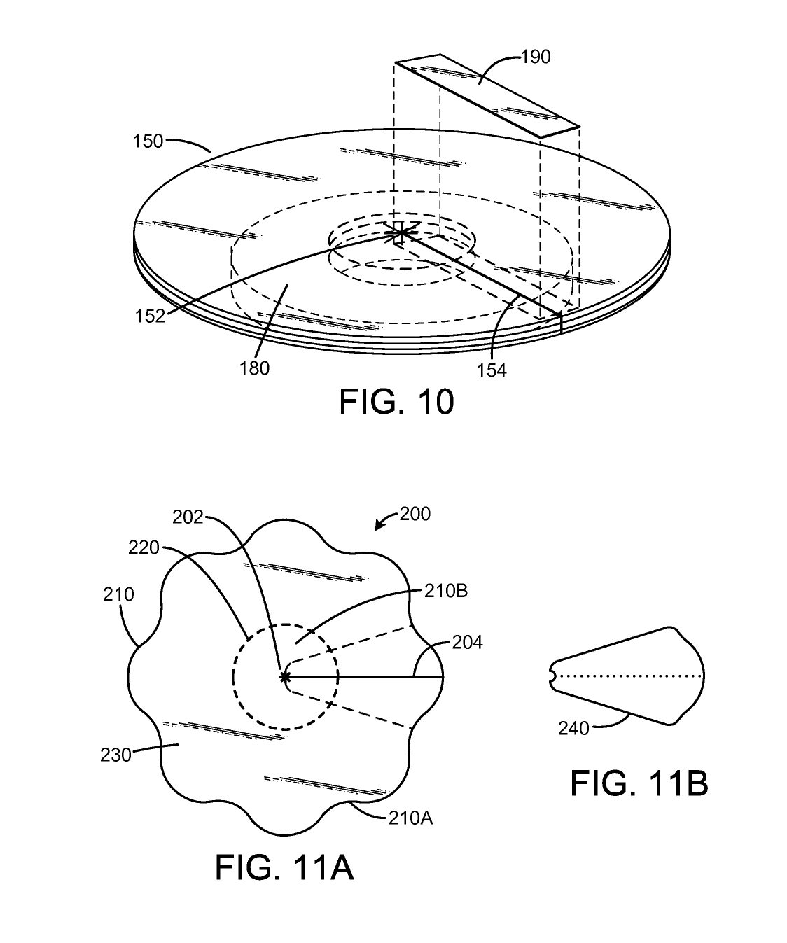

A further embodiment 150 of a wound dressing assembly is shown in FIG. 10. The dressing assembly is similar to assembly 50, except that the slit 104 formed through the assembly to permit the assembly to fit around the tube is generally linear from the tube port 152 to the assembly edge, and a separate seal 190 is used to cover the adjacent edges of the slit area after the assembly has been placed on the patient's body. The seal 190 may be a transparent adhesive tape length, for example.

In other embodiments, the dressing assembly may be formed without a slit, and the dressing applied by inserting a distal end of the tube through the tube port. This embodiment provides the nurse the option to cut a slit in the dressing if desired, e.g. by use of scissors or knife.

Exemplary sizes for the wound dressing may range from 1 inch diameter to 6 inches or larger, for round or capsule shapes (oblong or race-track shaped) from 1.5 inches to 6 inches or larger. The tube support diameters and dressing sizes/scales will be selected in dependence on the diameter of the tube or other device protruding from the body.

Materials for the wound dressing may vary, but will typically be latex free. Vinyl, cotton and non-woven fabrics may also be employed, for example. Parts of the dressing, such as portions of the cover layer, may be ventilated with perforation patterns. In other embodiments, the dressing may be fabricated as a co-injection molded assembly, wherein the absorbent material 80 is placed in the mold and bonded to the cover layer formed by the injection molding, using silicon or vinyl to form the cover layer. In this case, the adhesive to hold the dressing to the body is applied after the co-molding process.

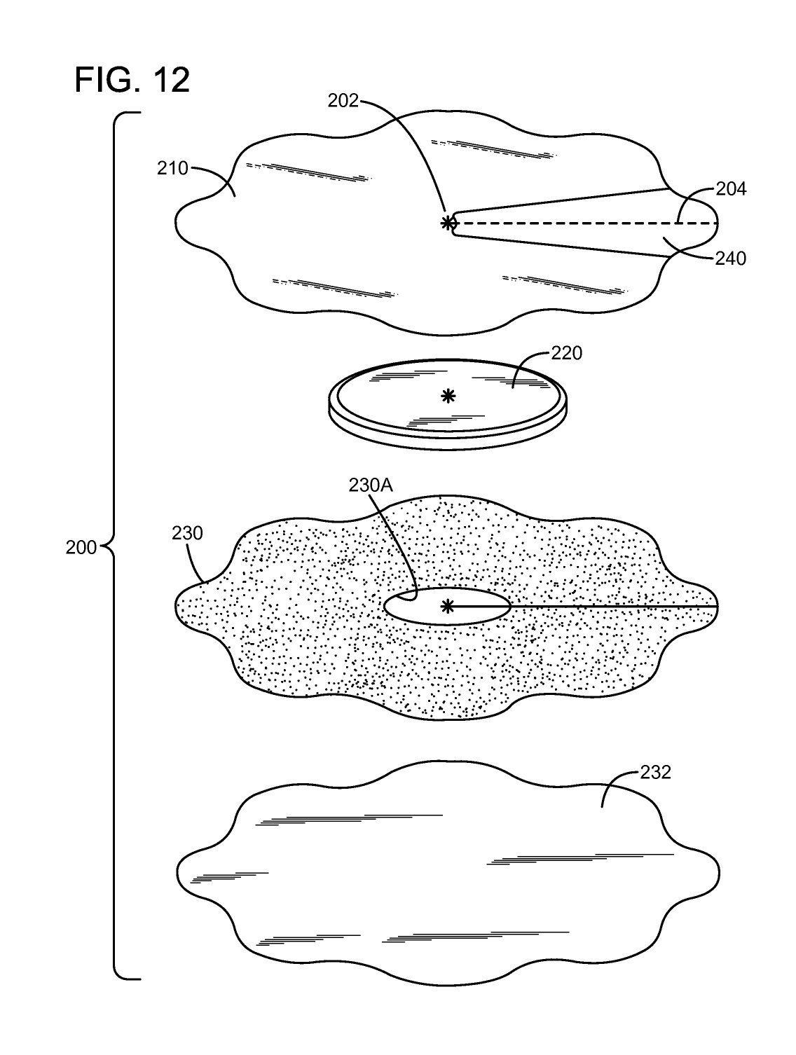

FIGS. 11A, 11B and 12 illustrate another embodiment of a wound dressing 200. In this embodiment the transparent or translucent cover layer 210 has an undulating perimeter 210A about a generally circular configuration. A slit 204 extends from the tube port 202 to the perimeter 210A, to allow the dressing to be fitted onto a patient's body with an existing tube in place. In this example, a separate seal 240 is employed to fit over the slit once the dressing has been placed on the patient's body. The seal 240 has a bottom layer of adhesive, covered by release strips which are removed before placing the seal onto the cover layer. An absorbent layer 220, in this embodiment, a hydrocolloid gel disc, is adhered to the bottom surface of the cover layer 210 by an adhesive layer 230. In this example, the layer 230 covers the entire bottom surface of the cover layer 210, and a release sheet or cover 232 covers the exposed adhesive until it is removed prior to use of the dressing. The hydrocolloid gel can be formulated to be more absorbent than cotton gauze, and in this embodiment is a translucent layer allowing the wound to be generally observable through the gel layer. The slit 204 is formed through the gel disc as well. In one exemplary embodiment, the dressing 200 is about 3 inches in diameter, and the absorbent layer is about 1 inch in diameter. The dressing may be made in larger or smaller sizes.

FIG. 12 is an exploded view of the dressing 200. In an exemplary embodiment, the absorbent layer 220 is fitted against the bottom surface of the cover layer 210, and aligned with the center 202. The hydrocolloid gel can have sufficient adhering properties to stick to the cover layer 210 without the need for a separate adhesive layer. An adhesive layer 230 is applied to the bottom surface of the cover layer outside the layer 220. In another embodiment, in which the layer 220 does not have sufficient stickiness to adhere to the cover sheet directly, the adhesive layer may first be applied to the entire bottom surface of the cover layer, and the absorbent layer 220 then applied to the adhesive layer. The adhesive can be, for example, a silicone or acrylic adhesive, for example. An adhesive cover layer or release sheet 232 is attached over the bottom of the dressing to cover the adhesive and layer 220, and is removable by the user prior to the application to the body.

The cover sheet 210 may be fabricated of a thin flexible translucent or transparent film, preferably a waterproof breathable film, e.g. 3 mil in thickness. Suitable films are commercially available, e.g. those used in dressing marketed as “Duoderm”, “Granuflex”, “Ultex” and “Tegaderm.” The absorbent layer 220 may have a thickness on the order of 18 mils, in an exemplary embodiment. Other thicknesses of the cover layer and absorbent layer may alternatively be employed.

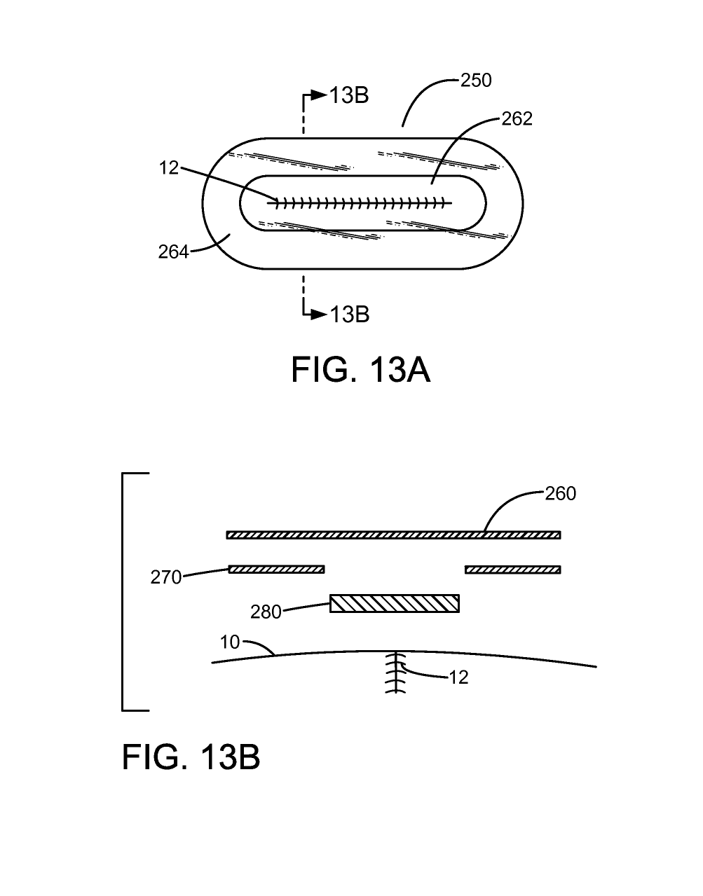

FIGS. 13A and 13B illustrate another embodiment of a wound dressing 250. In this embodiment, the dressing has an elongated oval or racetrack shape configuration, for covering a patient's wound on skin 10 closed by stitches 12. For other embodiments, different shape configurations may be employed, such as round, elliptical, rectilinear and the like. As with the embodiments described above, the dressing includes a thin transparent or translucent top cover layer 260, fabricated of a flexible material. A translucent or transparent absorbent pad 270 is disposed below the cover layer within a window 262. In one example, the pad 270 is a hydrocolloid gel pad. An adhesive layer 280 surrounds the absorbent pad on the underside of the layer 260, at peripheral region 264, and secures the dressing in place on the patient's skin 10. In this embodiment, a slit or opening is not formed in the cover layer 260, since no tube or other device need not be accommodated.

Although the foregoing has been a description and illustration of specific embodiments of the invention, various modifications and changes thereto can be made by persons skilled in the art without departing from the scope and spirit of the invention.

What is claimed is:

1. A surgical wound dressing assembly for a surgical tube wound or other device entrance into the body, comprising:
a cover layer, with adhesive on the outer perimeter of the underside of the cover layer;
an absorbent ring secured to the underside of the cover;
an interior clear window allowing the nurse or other medical personnel to view the wound site through the window for inspection for infection or bleeding; and
a port formed in the window area to allow a tube or other device to pass through the wound dressing.

2. The wound dressing assembly of claim 1, wherein the dressing is sliced from the center all the way to the outside allowing the nurse to apply the dressing to a wound site with an existing tube or other device exiting the human body, by opening or spreading the dressing apart at the slice or slit sufficiently to pass the dressing around the tube or other device.

3. The wound dressing assembly of claim 1, wherein the port is formed by forming short slits in the cover layer radially outwardly from a center.

4. The wound dressing assembly of claim 1, further comprising:
a tube support structure configured to fit over the dressing and support the tube or other device.

5. A wound dressing assembly for a tube or other device entrance into a patient's body at a wound site, comprising:
a cover layer having an outer perimeter and an underside, with adhesive at least on the outer perimeter of the underside of the cover layer;
an absorbent layer disposed at the underside of the cover and interior of the outer perimeter of the cover, and wherein at least an outer portion of the adhesive extends outside the absorbent layer;
an interior clear window allowing the nurse or other medical personnel to view the wound site through the window for inspection for infection or bleeding;
a port formed in the window area to allow the tube or other device to pass through the wound dressing; and
wherein the dressing is configured to be secured to the patient's body by the portion of the adhesive outside the absorbent layer.

6. The wound dressing assembly of claim 5, wherein the dressing has a slice extending from the port to the outer periphery, allowing the dressing to be applied to a wound site with an existing tube or other device exiting the patient's body, by opening or spreading the dressing apart at the slice sufficiently to pass the dressing around the tube or other device.

7. The wound dressing assembly of claim 5, wherein the port is formed by forming short slits in the cover layer radially outwardly from a center.

8. The wound dressing assembly of claim 5, further comprising:
a tube support structure configured to fit over the dressing and support the tube or other device.

9. The wound dressing assembly of claim 5, wherein the outer portion of the adhesive layer is covered with a cover sheet or release layer which is configured for removal before the dressing is placed onto the patient's body.

10. The wound dressing assembly of claim 9, wherein an interior portion of the adhesive is configured to hold in place the absorbent layer.

11. The wound dressing assembly of claim 5, wherein the absorbent layer is a gauze.

12. The wound dressing assembly of claim 11, wherein the absorbent layer has an interior opening positioned under the window.

13. The wound dressing assembly of claim 5, wherein the absorbent layer includes a hydrocolloidal gel.

14. The wound dressing assembly of claim 5, wherein the cover layer is fabricated of a flexible plastic material or film.

15. The wound dressing of claim 5, wherein the outer perimeter is undulating about a generally circular configuration.

16. The wound dressing assembly of claim 5, wherein the outer perimeter is circular.

17. A wound dressing assembly for application to a patient's body at a wound site, comprising:
a transparent or translucent cover layer having an outer perimeter and an underside;
an adhesive disposed at least on the outer perimeter of the underside of the cover layer;
an absorbent layer disposed at the underside of the cover and interior of the outer perimeter of the cover, and wherein at least an outer portion of the adhesive extends outside the absorbent layer;
the cover layer providing a transparent or translucent window allowing a nurse or other medical personnel to view the wound site through the window for inspection for infection or bleeding;
a removable adhesive cover layer disposed over the adhesive disposed on the outer perimeter of the underside of the cover layer; and
wherein the dressing is configured to be secured to the patient's body by the portion of the adhesive outside the absorbent layer after removal of the adhesive cover layer.

18. The wound dressing assembly of claim 17, further comprising:
a port formed in the window area to allow a tube or other device to pass through the wound dressing.

19. The wound dressing assembly of claim 17, wherein the absorbent layer includes a hydrocolloidal gel.

20. The wound dressing assembly of claim 17, wherein the cover layer is fabricated of a flexible, breathable plastic material or film.
